(19) United States
(12) Patent Application Publication
(10) Pub. No.: US 2010/0125321 A1
(43) Pub. Date: May 20, 2010

(54) EPTFE FILL OF COIL FILAR GAPS

(76) Inventors: Mark C. LYNN, Circle Pines, MN (US); Shrojalkumar Desai, Little Canada, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/576,706

(22) Filed: Oct. 9, 2009

Related U.S. Application Data
(60) Provisional application No. 61/114,577, filed on Nov. 14, 2008.

(51) Int. Cl. A61N 1/05; H01B 13/00

(52) U.S. Cl. 607/122; 607/115; 29/825

(57) ABSTRACT

A medical electrical lead includes a coiled electrode. In one example, the coiled electrode includes two conductive filars wound in parallel to define a plurality of turns and a gap between each turn. A polymeric filling is disposed in at least some of the gaps existing between each of the turns. The polymeric filling can include multiple layers of a thin polymeric film or a non-conductive, polymeric filar. The coiled electrode also includes a polymeric covering disposed over the outer surface of the electrode. The polymeric covering is bonded to the polymeric filling disposed in the gaps. The polymeric film includes a non-expanded polymer and the polymeric cover includes an expanded polymer. In some examples the non-expanded polymer is polytetrafluoroethylene (PTFE) and the non-expanded polymer is expanded polytetrafluoroethylene (ePTFE).

### CROSS-REFERENCE TO RELATED APPLICATION

This application claims the benefit under 35 U.S.C §119 of U.S. Provisional Application No. 61/114,577, filed on Nov. 14, 2008, entitled “EPTFE FILL OF COIL FILAR GAPS,” which is herein incorporated by reference in its entirety.

### TECHNICAL FIELD

The present invention relates to a medical electrical lead including one or more coiled electrodes. More particularly, the present invention relates to a coiled electrode including an electrically transparent cover.

### BACKGROUND

Medical electrical leads such as pacemakers and defibrillators may include a lead body having a coiled electrode that is implanted at an appropriate location within a patient's heart. An implantable defibrillator, for example, includes a lead assembly having at least one defibrillation electrode, such as a defibrillation coil. Some lead assemblies include a cover such as a polytetrafluoroethylene (PTFE) cover that extends over at least a portion of the outer surface of the coiled electrode. Such covers are used, for example, to prevent tissue ingrowth and to facilitate removal of the lead from the vessel in which it has been implanted. One challenge with such covers is that they may move during insertion of the lead through an introducer, potentially leaving a portion of the electrode exposed. This challenge may be heightened when the electrode coil is formed with spaces between turns of the coil to increase electrode flexibility, because the spaces tend to reduce the contact area between the electrode surface and the cover.

### SUMMARY

One embodiment of the present invention is a medical electrical lead including a lead body, at least one conductor, and at least one coiled electrode located on the lead body. The lead body includes a proximal end and a distal end. A terminal connector for connecting to a pulse generator or the like is located at the proximal end of the lead body. The conductor is coupled to the terminal connector and extends within the lead body from the proximal to the distal end. The coiled electrode is operatively coupled to the conductor extending within the lead body. The coiled electrode includes at least one wound conductive filar that defines an outer electrode surface including a plurality of gaps in the wound conductive filar. A polymeric filling including non-expanded polytetrafluoroethylene is disposed in and substantially fills at least some of the gaps. A polymeric cover including expanded polytetrafluoroethylene is disposed over the outer surface of the coiled electrode and is bonded to the polymeric filling provided in the gaps.

Another embodiment of the present invention is a method of forming an electrode. The method includes forming a coiled electrode including at least one conductive filar wound to define, in longitudinal cross-section, a plurality of turns and a gap between each turn. Additionally, the method includes filling at least a portion of the gaps with a polymeric filling comprising a non-expanded polytetrafluoroethylene; wrapping a cover comprising one or more layers of a thin polymeric film comprising expanded polytetrafluoroethylene over the outer surface of the electrode; and bonding the cover to the filling. In some embodiments, the polymeric filling includes one or more layers of a thin polymeric film comprising polytetrafluoroethylene. In other embodiments, the polymeric filling includes a filar comprising polytetrafluoroethylene. The cover can be sintered to the fillings disposed in the gaps.

According to another embodiment, a medical electrical lead includes an insulative lead body including a lumen through which a conductor extends and at least one coiled electrode located on the lead body and operatively coupled to the conductor. The coiled electrode includes at least one wound conductive filar that defines, along its longitudinal cross-section, a plurality of turns and a plurality of gaps disposed between the turns. A polymeric filling comprising polytetrafluoroethylene is disposed in and substantially fills at least some of the gaps. The filled gaps have a width of between about 0.0002 inches and about 0.0020 inches. A polymeric cover comprising expanded polytetrafluoroethylene is disposed over an outer surface of the coiled electrode and is bonded to the polymeric filling.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a partial cross-sectional view of a medical electrical lead including at least one coiled electrode according to various embodiments of the present invention.

FIGS. 2A-2D are longitudinal cross-sectional views of a portion of a lead body including a coiled electrode according to various embodiments of the present invention.

FIGS. 3A and 3B are longitudinal cross-sectional views of a coiled electrode including a polymeric filling according to various embodiments of the present invention.

FIGS. 4A and 4B are partial schematic views of a coiled electrode including a polymeric cover according to various embodiments of the present invention.

FIG. 5 is a flow chart of a method according to an embodiment of the present invention.

FIG. 6 is a flow chart of a method according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

Embodiments of the present invention are directed to a medical electrical lead. According to some embodiments, the medical electrical lead can be configured for implantation within a patient's heart. According to other embodiments, the medical electrical lead is configured for implantation within a patient's neurovascular regions.

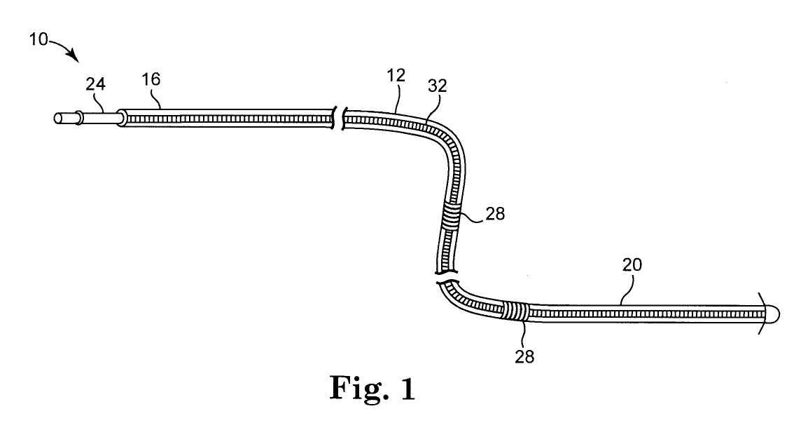

FIG. 1 illustrates a defibrillation lead 10, which includes an elongated, insulative lead body 12 extending from a proximal end 16 to a distal end 20. The proximal end 16 is configured to be operatively connected to a pulse generator via a connector 24. A conductor 32 extends within the lead body 12 from the connector 24 to at least one coiled electrode 28 located on the lead body 12. The lead body 12 can also include one or more fixation members for securing and stabilizing the lead body 12 including the one or more electrodes 28 at a target site within a patient's body. The fixation member(s) can be active or passive.

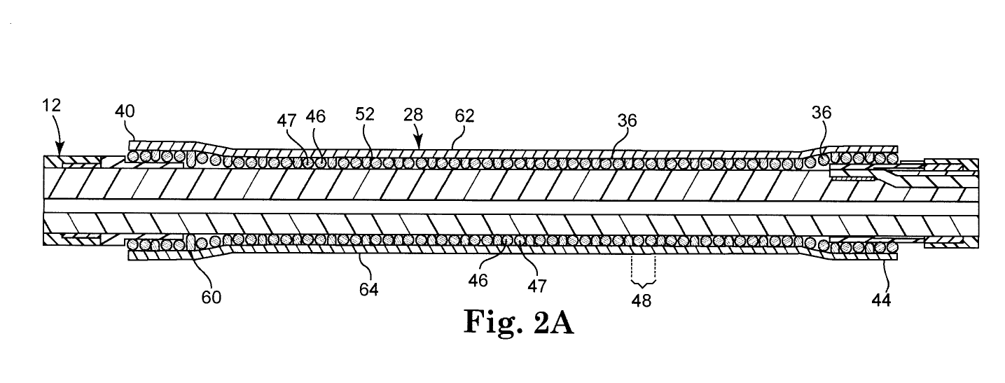

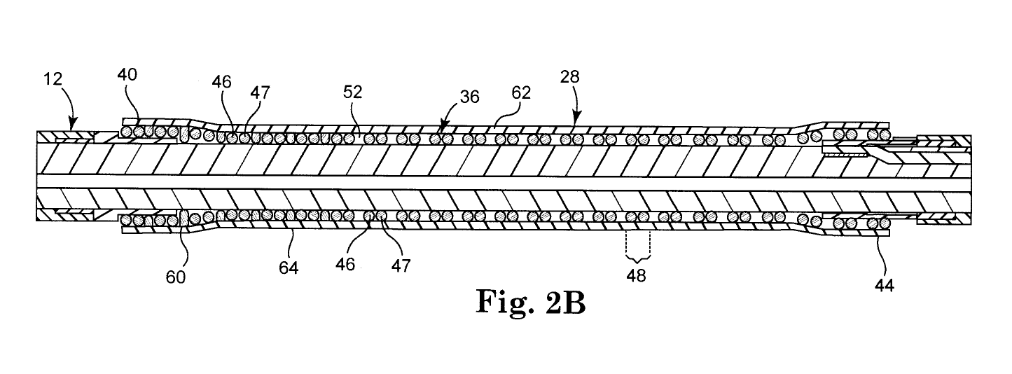

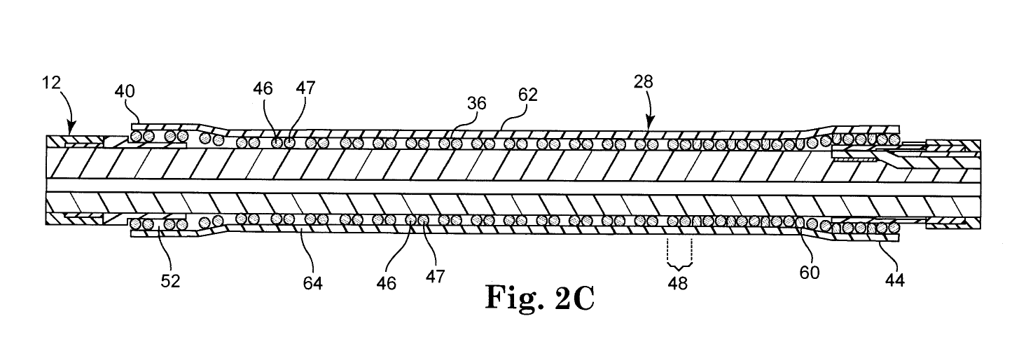

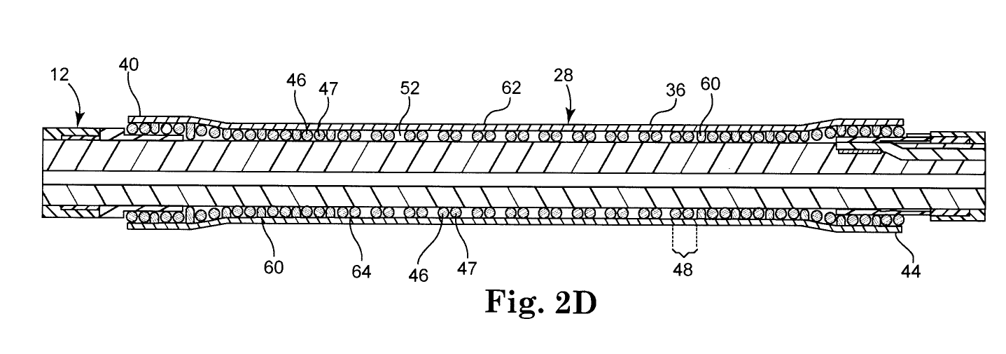

FIGS. 2A-2D are longitudinal cross sectional views of a portion of the lead body 12 including the coiled electrode 28 according to various embodiments of the present invention. The coiled electrode 28 includes an outer surface 36 and extends from a first end 40 to a second end 44. According to various embodiments of the present invention, the coiled electrode 28 is formed from at least one conductive filar 46. In some embodiments, the coiled electrode 28 is formed from a plurality of conductive filars 46.

According to one exemplary embodiment of the present invention, as shown in FIGS. 2A-2D, the coiled electrode 28 is formed from two conductive filars 46, 47 wound in parallel to define a plurality of turns 48 and a gap 52 between each turn. A polymeric filling 60 is disposed in at least some of the gaps 52 existing between each of the turns 48. The coiled electrode 28 also includes a polymeric covering 64 disposed over the outer surface 36 of the electrode and extending from the first end 40 to the second end 44 of the electrode 28. The polymeric covering 64 is bonded to the polymeric filling 60 disposed in the gaps 52. According to other embodiments, the polymeric cover 64 may extend beyond the electrode 28 and cover at least a portion of the lead body 12 in addition to the electrode 28.

The polymeric filling 60 can be disposed in some or all of the gaps between each of the turns 48 of the coiled electrode 28. The gaps 52 between the conductive filars 46, 47 are sufficiently wide so as to receive the polymeric filling 60 disposed therein. Additionally, the gaps 52 are sufficiently wide to maintain flexibility of the electrode 28. Flexibility is an important feature of coiled defibrillation electrodes. In general, the gap width can be represented by the following mathematical expression:

gap width=wire pitch−(no. of filars×filar diameter)

The wire pitch is the distance in the longitudinal direction that a single filar covers in one rotational wind. According to one embodiment, a width of the gaps 52 ranges from about 0.0002 to about 0.020 inches. According to another embodiment, the gap width is about 0.0010 inches.

In some embodiments, as shown in FIG. 2A, the polymeric filling 60 is disposed in substantially all of the gaps 52 extending from the first end 40 to the second end 44 of the electrode 28. In other embodiments, as shown in FIGS. 2B and 2C, the polymeric filling 60 is disposed in a portion of the gaps 52 located at either the first end 40 (FIG. 2B) or the second end 44 (FIG. 2C) of the electrode 28. In still other embodiments, as shown in FIG. 2D, the polymeric filling 60 is disposed in a portion of the gaps 52 located at both the first end 40 and the second end 44 of the electrode 28. In this particular example, the polymeric filling 60 is not disposed in the gaps 52 in the middle portion 62 of the electrode 28.

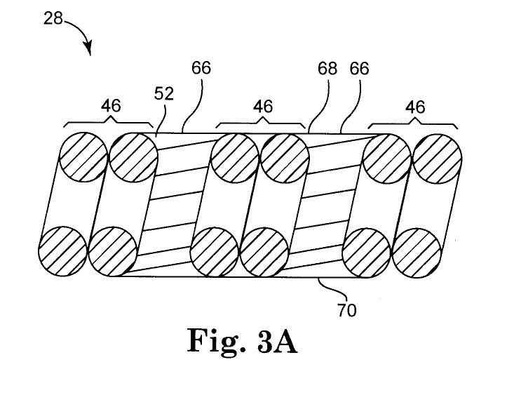

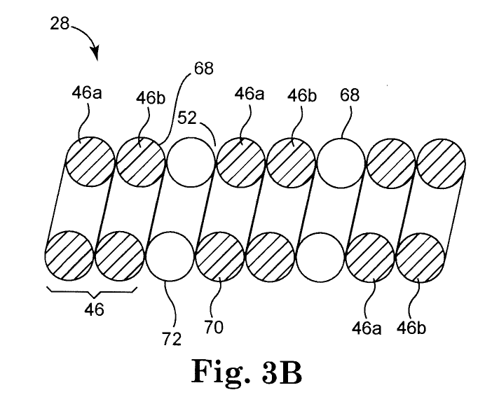

According to some embodiments of the present invention, the polymeric filling 60 includes one or more layers of a polymeric film 66. FIG. 3A is a longitudinal cross-sectional view of a bifilar coiled electrode 28 including multiple layers of a polymeric film 66 disposed in the gaps 52 between the conductive filars 46, 47. The polymeric film 66 has a width equal to or less than the width of the gap 52 between each of the turns 48 of the coiled electrode 28. The polymeric film 66 is wound into the desired gaps 52 of the coiled electrode 28 such that the gaps 52 are substantially filled with the polymeric film 66. As shown in FIG. 3A, the filling 60 extends in the gap 52 between each conductive filar (or group of filars) from essentially a top surface 68 to a bottom surface 70 of the coiled electrode 28. Multiple layers of the polymeric film 66 may be necessary to substantially fill the desired gaps 52. The film 66 can be wrapped about the electrode 28 such that at least some of the gaps 52 are filled, as shown in FIGS. 2A-2D.

According to other embodiments of the present invention, the polymeric filling 60 includes a non-conductive, non-porous polymeric filar 72. FIG. 3B is a longitudinal cross-section of a bifilar coiled electrode 28 including two conductive filars 46, 47 and a non-conductive filar 68. In one embodiment, the polymeric filar 72 is co-wound with the conductive filars 46, 47 during fabrication of the coiled electrode 28. In another embodiment, the polymeric filar 72 is wound into the desired gaps 52, between the conductive filars 46, as shown in FIGS. 2A-2D, after the electrode 28 has been fabricated. The polymeric filar 72 substantially fills the desired gaps 52 between the conductive filars 46. As best shown in FIG. 3B, the polymeric filar 72 is wound such that a longitudinal cross-section of the coiled electrode 28 shows a repeating pattern of a first conductive filar 46a directly adjacent to the second conductive filar 46b. The polymeric filar 72 is directly adjacent to the second conductive filar 46b.

As shown in FIGS. 2A-2D, the polymeric cover 64 is disposed over the outer surface 36 of the coiled electrode 28 from substantially the first end 40 to the second end 44 of the electrode 28. In certain embodiments, the polymeric cover 64 may extend beyond one or both ends 40, 44 of the electrode 28 and over at least a portion of the lead body 12. According to various embodiments, the polymeric cover 64 may include one or more layers of a thin polymeric film. In some embodiments, the polymeric cover 64 can include as many as 120 layers of a thin polymeric film 74. The resulting polymeric cover 64 can have a thickness ranging from about 1 to about 25 microns.

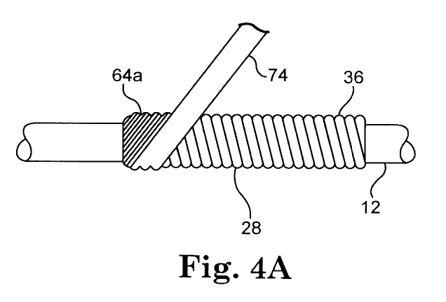

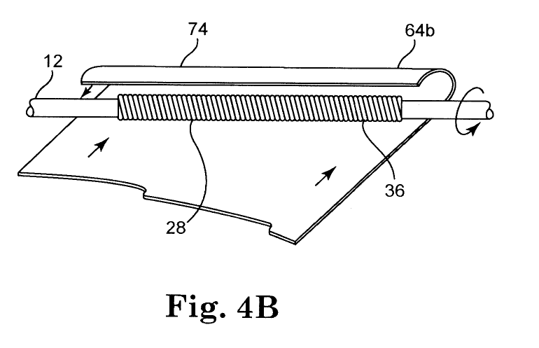

The polymeric film 74 may be wrapped about the outer surface 36 of the electrode 28 to form the polymeric cover 64 according to a variety of methods. An exemplary film wrapping process is shown and described in U.S. Pat. No. 7,020,529 entitled “Defibrillation Electrode Cover” the description of which is incorporated herein by reference. FIG. 4A is a partial schematic view of a lead body 12 including a coiled electrode 28 having a helically wrapped polymeric cover 64a. FIG. 4B is a partial schematic view of a portion of a lead body 12 including a coiled electrode 28 having a cylindrically wrapped polymeric cover 64b.

According to various embodiments of the present invention, the polymeric filling 60 and the polymeric cover 64 can be fabricated from structurally similar polymers having different material properties. According to various embodiments, the polymeric filling 60 is formed from a first polymeric material having a first set of material properties and the polymeric cover 64 is formed from a second polymeric material having a second set of material properties. The first polymeric material used to fabricate the filling 60 may differ in dielectric strength, porosity, and/or linear strength from the second polymeric material used to form the polymeric cover 64. In some embodiments, for example, the polymer filling 60 includes a polymer of a higher dielectric strength than the polymer used to form the polymer cover 64. In other embodiments, the polymer filling includes an essentially non-porous polymeric material or a polymeric material having a low degree of porosity and the polymer cover includes a porous polymeric material. In certain embodiments, the porous polymeric material has sufficient porosity to promote conductivity.

According to other embodiments of the present invention, the polymer filling 60 includes a non-expanded polymer and the polymer cover 64 includes an expanded polymer. The non-expanded polymer used to form the filling 60 has a higher dielectric strength than the expanded version of the same polymer. Additionally, the non-expanded polymer is essentially non-porous or has a lower porosity than the expanded polymer. The non-porous characteristics of the non-expanded polymer makes it unable to support conductivity. In contrast to the non-expanded polymer, the expanded polymer has a degree of porosity that is large enough to support conductivity when wetted with an appropriate ionic fluid, but small enough to prevent tissue ingrowth.

According to one embodiment, the polymer filling includes a non-expanded version of the same polymer used to make the polymer cover. Varying forms of the same polymer, or two polymers with structurally similar chemical backbones bond well to one another. A polymeric cover 64 that is strongly bonded to the polymeric filling 60 may be less likely to shift during implantation of the electrode. Thus, the potential for a portion of the electrode becoming exposed during implantation can be minimized. Minimizing exposure of the coiled electrode prevents tissue ingrowth. The prevention of tissue ingrowth into the coiled electrode is an important factor in facilitating removal of the lead from the implanted location.

Suitable biocompatible polymers that can be used to fabricate the polymeric filling 60 and the polymeric cover 64 include non-expanded and expanded versions of the following exemplary polymers, included but limited to, the following: polyethylene (PE), polypropylene (PP), fluorinated ethylene propylene (FEP), ethylene-tetrafluoroethylene (ETFE), polytetrafluoroethylene (PTFE), or suitable biocompatible polymers known to those of skill in the art.

According to one embodiment, the polymeric filling 60 is fabricated from PTFE and the polymeric cover 64 is formed from expanded polytetrafluoroethylene (ePTFE). The PTFE used to form the filling can be essentially non-porous and thus serves as an insulator in the gaps between the coil turns. The ePTFE used to form the cover 64 can be fabricated such that is has a degree of porosity sufficient to support conductivity, but small enough to prevent tissue ingrowth.

According to various embodiments of the present invention, the polymeric cover 64 is bonded to the polymeric filling 60 disposed in the gaps 52 between the turns 48 of the coiled electrode 28. In some embodiments, the polymeric cover is covalently bonded to the polymeric filling. The cover 64 may be bonded to the polymer filling 60 using a variety of methods including heat bonding, solvent bonding, or laser sintering. According to one embodiment, the cover 64 is sintered to the polymeric filling 60 using a laser, infrared (IR) gun, heat gun, or cover.

PTFE and ePTFE can be made to covalently bond to one another using surface modification techniques followed by using an adhesive tie-layer to covalently bond the two materials. In one embodiment, heat fusion can also be used to bond the ePTFE material to the PTFE material. In another embodiment, the surface of the conductive filar can be treated using plasma treatment techniques to provide a fluorocarbon containing coating. A fluorocarbon containing coating allows the fluoropolymer to flex in the same manner as the conductive filar. Exemplary fluorocarbon plasmas used to treat the surface of the conductive filar include, but are not limited to: fluoro ethylene propylene, perfluoropropane, and octafluorocyclobutane. The fluorocarbon containing coating provided on the surface of the conductive filar can be made to fuse with the fluorocarbon filling (e.g. PTFE, ePTFE, or another similar material) via heat fusion causing the polymer chains to physically interlock via Van der Waals interactions. This will enhance the adhesion between the plasma coated filar and the polymer filling. In yet another embodiment, the surface of the polymer filling is treated using chemical stripping using, for example, sodium naphthalene/argon or plasma etching to remove the fluorine groups from the polymeric material followed by applying a medical adhesive to a surface of the conductive filar and polymeric filling to bond the two materials together.

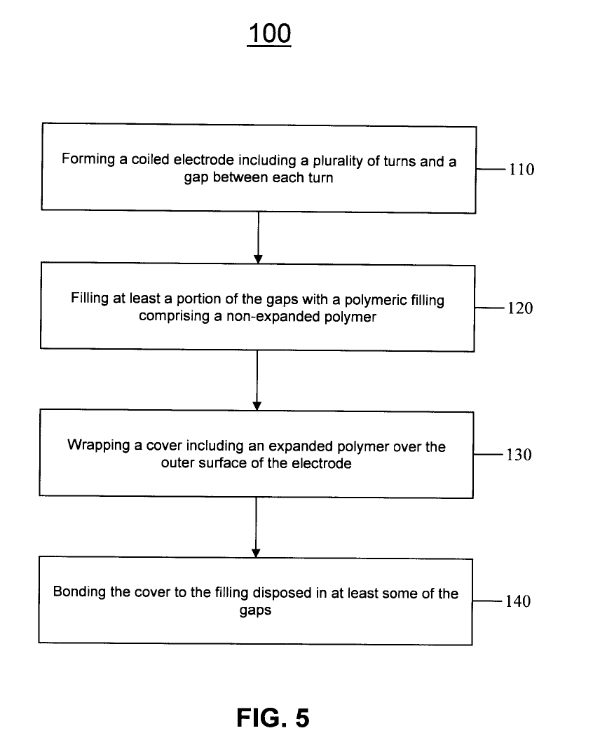

FIG. 5 is a flow chart of a method (100) used to fabricate a coiled electrode according to an embodiment of the present invention. First, a coiled electrode is formed by winding one or more conductive filars to form a coil (Block 110). The coiled electrode includes a plurality of turns and a gap existing between each turn. Each turn can include a single filar or a group of filars. In one embodiment, the coiled electrode is a bifilar coiled electrode with a gap existing between every two conductive filars. Next, according to one embodiment, at least a portion of the gaps are filled with a polymeric filling (Block 120). In certain embodiments, the polymeric filling includes a non-expanded polymer. In one embodiment, the non-expanded polymer is polytetrafluoroethylene. According to one embodiment, the gaps can be filled by wrapping a thin polymeric film, including a non-expanded polymer, into the gaps until the gaps are substantially filled. Multiple passes with a thin polymeric film may be required to substantially fill the gaps. According to another embodiment, a polymeric filar may be wound into the gaps. The polymeric filar should be sufficiently wide so as to substantially fill the gaps. After the gaps have been filled, a polymeric cover including one or more layers of a thin polymeric material, including an expanded polymer is wrapped about the outer surface of the coiled electrode (Block 130). In certain embodiments, the expanded polymer is expanded polytetrafluoroethylene (ePTFE). A helical wrap or a cylindrical wrap may be employed. Multiple layers of the polymeric film may be wrapped about the outer surface of the electrode to achieve a desired thickness. The cover is then bonded to the polymeric filling disposed in the gaps (Block 140). In certain embodiments, the cover is laser sintered to the polymeric filling disposed in the gaps.

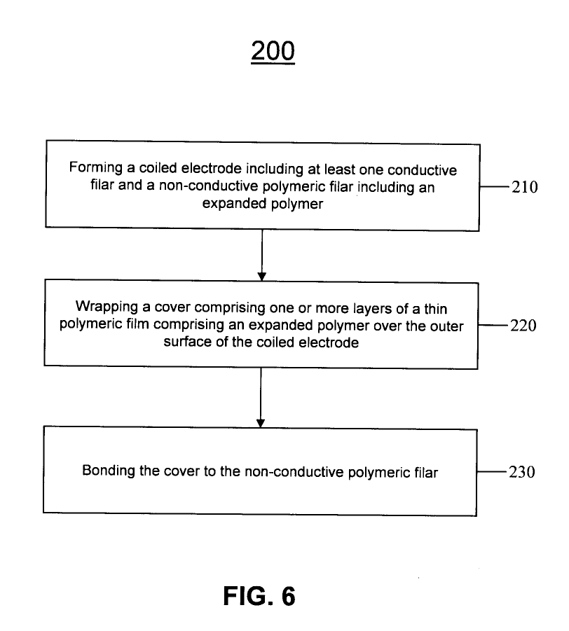

FIG. 6 is a flow chart of a method (200) according to another embodiment of the present invention. According to this embodiment, a coiled electrode is formed including at least one conductive filar and a polymeric filar including non-expanded polymer. In certain embodiments, the coiled electrode includes two conductive filars. The conductive filar(s) and the polymeric filar are wound together simultaneously such that a longitudinal cross-section of the coiled electrode reveals a repeating pattern of a first conductive filar directly adjacent to a second conductive filar, directly adjacent to the polymeric filar (Block 210). After the electrode has been formed including the polymeric filar, a polymeric cover including one or more layers of a thin polymeric film including an expanded polymer is wrapped about the outer surface of the coiled electrode (Block 220). A helical wrap or a cylindrical wrap may be employed. Multiple layers of the polymeric film may be wrapped about the outer surface of the electrode to achieve a desired thickness. The cover is then bonded to the polymeric filling disposed in the gaps (Block 230). In certain embodiments, the cover is laser sintered to the polymeric filling disposed in the gaps.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A medical electrical lead comprising:
a lead body including a proximal end, a distal end, and a terminal connector located at the proximal end;
at least one conductor coupled to the terminal connector and extending within the lead body from the proximal end to the distal end;
at least one coiled electrode located on the lead body and operatively coupled to the at least one conductor, the coiled electrode including at least one wound conductive filar that defines an outer electrode surface, the outer electrode surface including a plurality of gaps in the wound conductive filar;
a polymeric filling comprising non-expanded polytetrafluoroethylene disposed in and substantially filling at least some of the plurality of gaps; and
a polymeric cover comprising an expanded polytetrafluoroethylene disposed over the outer surface of the coiled electrode and bonded to the polymeric filling.

2. The lead according to claim 1, wherein the coiled electrode is a defibrillation electrode.

3. The lead according to claim 1, wherein the polymeric filling is disposed in a plurality of gaps disposed at the first end, the second end, or both the first and second ends of the coiled electrode.

4. The lead according to claim 1, wherein the polymeric filling comprises one or more layers of a thin film comprising polytetrafluoroethylene (PTFE).

5. The lead according to claim 1, wherein the polymeric filling comprises a polymeric filar comprising polytetrafluoroethylene (PTFE).

6. The lead according to claim 1, wherein the polymeric cover includes one or more layers of a helically wrapped film comprising expanded polytetrafluoroethylene (ePTFE).

7. The lead according to claim 1, wherein the polymeric cover includes one or more layers of a cylindrically wrapped film comprising expanded polytetrafluoroethylene (ePTFE).

8. The lead according to claim 1, wherein the gaps are sufficiently wide so as to receive the polymeric filling disposed therein such that a longitudinal cross section of the coiled electrode includes the polymeric filling in the gaps extending from a top surface to a bottom surface of the electrode.

9. A medical electrical lead comprising:
an insulative lead body including at least one lumen through which a conductor extends;
at least one coiled electrode located on the lead body and operatively coupled to the conductor, the coiled electrode including at least one wound conductive filar that defines, along its longitudinal cross-section, a plurality of turns and a plurality of gaps disposed between the turns;
a polymeric filling comprising polytetrafluoroethylene disposed in and substantially fills at least some of the gaps, the filled gaps having a width of between about 0.0002 inches and about 0.0020 inches; and
a polymeric cover comprising polytetrafluoroethylene disposed over an outer surface of the coiled electrode and bonded to the polymeric filling.

10. The lead according to claim 9, wherein the coiled electrode comprises a first wound conductive filar and a second wound conductive filar, wherein the longitudinal cross-section of the coiled electrode defines a repeating pattern of a first conductive filar turn and a second conductive filar turn directly adjacent to the first filar turn and a gap having a width of between about 0.0002 inches to about 0.0020 inches.

11. The lead according to claim 9, wherein the polymeric filling comprises a non-conductive polytetrafluoroethylene filar.

12. The lead according to claim 9, wherein the polymeric filling comprises a plurality of layers of a non-conductive polytetrafluoroethylene film.

13. A method comprising:
forming a coiled electrode extending from a first end to a second end and having an outer surface, the coiled electrode comprising at least one conductive filar wound to define, in longitudinal cross-section, a plurality of turns and a gap between each turn;
filling at least a portion of the gaps with a polymeric filling comprising a non-expanded polytetrafluoroethylene;
wrapping a cover comprising one or more layers of a thin polymeric film comprising an expanded polytetrafluoroethylene over the outer surface of the electrode; and
bonding the cover to the filling disposed in at least some of the gaps.

14. The method according to claim 13, wherein the step of filling at least a portion of the gaps comprises winding one or more layers of a polymeric film comprising polytetrafluoroethylene (PTFE) into the gaps such that the gaps are substantially filled.

15. The method according to claim 13, wherein the step of filling at least a portion of the gaps comprises winding a non-conductive polymeric filar comprising polytetrafluoroethylene (PTFE) into at least a portion of the gaps.

16. The method according to claim 13, wherein the step of bonding the cover to the filling comprises sintering the cover to the filling.

17. The method according to claim 13, further comprising treating the outer surface of the coiled electrode with a fluorocarbon plasma.

18. The method according to claim 13, wherein the fluorocarbon plasma comprises any one of fluoro ethylene propylene, perfluoropropane, and octafluorocyclobutane.

19. The method according to claim 13, further comprising chemically etching the polymer filling and applying a medical adhesive to at least the outer surface of the conductive filar.

20. The method according to claim 13, further comprising plasma etching the polymer filling and applying a medical adhesive to at least the outer surface of the conductive filar.
